(19) United States
(12) Patent Application Publication
(10) Pub. No.: US 2010/0305620 A1
(43) Pub. Date: Dec. 2, 2010

(54) MULTIDIRECTIONAL BONE FIXATION ASSEMBLY

(76) Inventor: Yechiel Gotfried, Kiryat Motzkin (IL)

Correspondence Address: HOLTZ, HOLTZ, GOODMAN & CHICK PC, 220 Fifth Avenue, 16TH Floor, NEW YORK, NY, 10001-7708, US

(21) Appl. No.: 12/599,805

(22) PCT Filed: May 18, 2008
(86) PCT No.: PCT/IL08/00678

§ 371 Date: Nov. 11, 2009

Related U.S. Application Data
(60) Provisional application No. 60/930,533, filed on May 17, 2007.

(51) Int. Cl. A61B 17/86; A61B 17/84

(52) U.S. Cl. 606/305; 606/328

(57) ABSTRACT

Apparatus is provided, including at least one double-jointed bone fixation element. A rod is coupled to the bone fixation element. A rod retaining portion retains the rod in place with respect to the bone fixation element. Other embodiments are also described.

### CROSS-REFERENCES TO RELATED APPLICATIONS

The present patent application claims priority from U.S. Provisional Patent Application 60/930,533 to Gotfried, filed May 17, 2007, entitled, “Multidirectional bone fixation assembly,” which is incorporated herein by reference.

### FIELD OF THE INVENTION

The present invention relates in general to implantable medical devices, and specifically to apparatus and methods for bone fixation.

### BACKGROUND OF THE INVENTION

The following patents, which are incorporated herein by reference, may be of interest:

U.S. Pat. No. 5,207,678 to Harms et al.

U.S. Pat. No. 5,474,555 to Puno et al.

### SUMMARY OF THE INVENTION

In some embodiments of the present invention, apparatus is provided, comprising a plurality of double-jointed bone fixation elements, which are each coupled to a rod. The double jointed nature of the bone fixation elements typically facilitates this coupling, while reducing bending of the rod. Typically, a method for using the apparatus comprises inserting a longitudinal bone insertion element such as a screw or a pin into a bone, and rotating a joint element to a desired orientation thereof, the joint element being coupled to the longitudinal bone insertion element. A rod holder for holding the rod is coupled to the joint element, and a desired angular orientation of the rod holder is identified. The rod holder is positioned at the desired angular orientation, and the rod is coupled to the rod holder when the rod holder is at the desired angular orientation, thereafter being tightened, such that the double-jointed bone fixation elements maintain the desired angular orientation.

There is therefore provided, in accordance with an embodiment of the invention, apparatus, including:

at least one double-jointed bone fixation element;

a rod, configured to be coupled to the bone fixation element; and

a rod retaining portion, configured to retain the rod in place with respect to the bone fixation element.

In an embodiment, the at least one double-jointed bone fixation element includes a plurality of double-jointed bone fixation elements, configured to be coupled to the rod.

In an embodiment, the apparatus further includes a single-jointed bone fixation element, configured to be coupled to the rod.

In an embodiment, the rod retaining portion includes at least one ring slidably coupled to the rod, configured to slide along the rod into a position that retains the rod in place with respect to the bone fixation element. Alternatively or additionally, the rod retaining portion includes a screw.

In an embodiment, the apparatus includes:

a longitudinal element configured for insertion into bone of a subject; and

a locking pin, configured to fix a position of the rod with respect to the longitudinal element.

In an embodiment,

the double-jointed bone fixation element defines first and second joints,

the double-jointed bone fixation element includes:
a mobile aperture element; and
a longitudinal bone insertion element configured to be inserted through the aperture element into bone, and

wherein at the first joint, the aperture element is configured to rotate with respect to the longitudinal bone insertion element.

In an embodiment,

the double-jointed bone fixation element includes a retaining connector, coupled to the mobile aperture element, shaped to define a support lip,

the mobile aperture element is shaped to define an interface lip thereof, and

the interface lip is configured to be in contact with the support lip.

In an embodiment, the interface lip is configured to articulate with the support lip.

In an embodiment, the longitudinal bone insertion element includes a screw. In an embodiment, the longitudinal bone insertion element includes a pin.

In an embodiment,

at the first joint, the mobile aperture element is configured to rotate such that a longitudinal axis of the mobile aperture element comes in alignment with a longitudinal axis of the longitudinal bone insertion element, and further rotates along a different axis to diverge from a longitudinal axis of the longitudinal bone insertion element,

the apparatus includes a retaining connector and a rod holder, configured to hold the rod, and

at the second joint, the retaining connector and the mobile aperture element facilitate further rotation of the rod relative to the bone fixation element by being rotated.

In an embodiment,

at the first joint, the mobile aperture element is configured to rotate such that a longitudinal axis of the mobile aperture element diverges from a longitudinal axis of the longitudinal bone insertion element,

the apparatus includes a retaining connector and a rod holder, configured to hold the rod, and

at the second joint, the retaining connector and the mobile aperture element facilitate further rotation of the rod relative to the bone fixation element by being rotated.

In an embodiment, the rod holder and the rod retaining portion are formed as an integral structure.

In an embodiment,

the double-jointed bone fixation element includes a retaining connector, rotatably coupled to the mobile aperture element,

at the second joint, the retaining connector is configured to rotate around the longitudinal axis of the mobile aperture element,

the apparatus includes a rod holder, configured to hold the rod,

the rod holder is configured to be rotated to facilitate placement of the rod in a desired alignment, and
the rotation of the retaining connector at the second joint is configured to maintain the desired alignment of the rod holder.

In an embodiment,

at the first joint, the mobile aperture element is configured to rotate such that a longitudinal axis of the mobile aperture element diverges from a longitudinal axis of the longitudinal bone insertion element,

the bone fixation element includes a retaining connector, rotatably coupled to the mobile aperture element, and

at the second joint, the retaining connector is configured to rotate around the longitudinal axis of the mobile aperture element.

In an embodiment, prior to the diverging of the longitudinal axes, the mobile aperture element is rotatable around the longitudinal axis of the bone insertion element.

There is further provided, in accordance with an embodiment of the invention, a method, including:

inserting a longitudinal bone insertion element into a bone;

rotating a double-jointed element to a desired orientation thereof, the double-jointed element being coupled to the longitudinal bone insertion element;

coupling a rod holder to the double-jointed element;

identifying a desired angular orientation of the rod holder;

positioning the rod holder at the desired angular orientation;

coupling a rod to the rod holder; and

reducing motion among the rod holder, the longitudinal bone insertion element, and the rod by tightening the double-jointed element.

In an embodiment, the motion includes reducing the motion via a locking pin, coupled at one end thereof to the rod holder and the rod, and at another end thereof to the longitudinal bone insertion element.

There is yet further provided, in accordance with an embodiment of the invention, a method, including:

inserting at least a first and a second longitudinal bone insertion element into respective bone sites of a patient, at different angular orientations of insertion;

coupling the first and second longitudinal bone insertion elements to respective first and second rod holders;

reducing an angular divergence between the rod holders by (a) manipulating a first double-jointed element that is coupled to the first rod holder and to the first longitudinal bone insertion element, and (b) manipulating a second double-jointed element that is coupled to the second rod holder and to the second longitudinal bone insertion element;

coupling the first and second double-jointed elements via a rod; and

reducing motion among the first longitudinal bone insertion element, the second longitudinal bone insertion element, and the rod, by tightening the double-jointed elements.

For some applications, the second double-jointed element referred to in step (b) is, instead, a jointed element that is not double-jointed.

In an embodiment, reducing the motion includes reducing the motion via a locking pin, coupled at one end thereof to the first rod holder and the rod, and at another end thereof to the first longitudinal bone insertion element.

There is still further provided, in accordance with an embodiment of the invention, apparatus for treating bone, including:

at least two bone anchoring members;

at least two rod holders;

coupled to each rod holder, an integrated double articulation element that (a) provides mobility of the rod holder by being adapted to move in at least two axes relative to one of the bone anchoring members at a first joint, and (b) provides further mobility of the rod holder relative to the one of the bone anchoring members at a second joint;

for each rod holder, at least one rod retaining portion; and

a connecting rod, configured to be coupled to each of the rod holders.

There is also provided, in accordance with an embodiment of the invention, a method for treating bone, including:

a. providing a bone fixation system, including:
1. at least two bone anchoring members;
2. at least two rod holders;
3. coupled to each rod holder, an integrated double articulation element that (a) provides mobility of the rod holder by being adapted to move in at least two axes relative to one of the bone anchoring members at a first joint, and (b) provides further mobility of the rod holder relative to the one of the bone anchoring members at a second joint;
4. for each rod holder, at least one rod retaining portion; and
5. a connecting rod, configured to be coupled to each of the rod holders;

b. inserting the at least two bone anchoring members in at least one bone;

c. coupling each bone anchoring member to a respective one of the rod holders;

d. bringing each of the rod holders, via its respective integrated double articulation element, to an orientation suitable for coupling the rod holder to the connecting rod;

e. coupling the connecting rod to each of the rod holders via at least one rod retaining portion; and

f. tightening each of the rod holders to minimize motion between each rod holder and the respective bone anchoring member and to minimize motion between the connecting rod and each of the rod holders.

There is additionally provided, in accordance with an embodiment of the invention, apparatus, including:

a rod;

a rod holder shaped to define an opening through which the rod passes;

a longitudinal element configured for insertion into bone of a subject;

a jointed element which (a) facilitates motion of the rod relative to the longitudinal element, (b) is shaped to define a space therein configured for holding the longitudinal element, and (c) is configured to reduce a distance between a center of the jointed element and a center of the rod holder; and

a locking pin having first and second ends, the first end pushing the rod against the rod holder when the rod is in the opening, and the second end pushing a lateral portion of the longitudinal element against a portion of the jointed element.

In an embodiment, the jointed element includes a coupling mechanism selected from the group consisting of: a threaded coupling mechanism, a ratchet coupling mechanism, and a click-into-place coupling mechanism, and the selected coupling mechanism facilitates the reduction in distance between the center of the jointed element and the center of the rod holder.

In an embodiment, the apparatus includes a plurality of longitudinal elements, each configured for insertion into bone of a subject.

In an embodiment, the apparatus includes a plurality of rods.

In an embodiment, (a) the first end of the locking pin pushes the rod against the rod holder and the second end of the locking pin pushes the lateral portion of the longitudinal element against the portion of the jointed element, in response to (b) the reduction in distance between the rod and the longitudinal element.

In an embodiment, the jointed element includes a mobile aperture element and a retaining connector.

In an embodiment, the jointed element includes a first joint element shaped to define a first lip and a second joint element shaped to define a second lip, the first and second lips configured to articulate with each other.

The present invention will be more fully understood from the following detailed description of embodiments thereof, taken together with the drawings, in which:

### BRIEF DESCRIPTION OF THE DRAWINGS

FIGS. 1, 2, and 3A-C are schematic illustrations of double-jointed apparatus for bone fixation, in accordance with embodiments of the present invention;

FIGS. 4 and 5 are schematic illustrations of the apparatus of FIG. 1, implanted in bone of a subject, in accordance with respective embodiments of the present invention;

FIG. 6 is a schematic illustration of apparatus like the apparatus of FIGS. 2 and 3A, implanted in bone of a subject, in accordance with an embodiment of the present invention;

FIG. 7 is a schematic illustration of apparatus like the apparatus of FIGS. 2 and 3B, implanted in bone of a subject, in accordance with an embodiment of the present invention; and

FIGS. 8 and 9 are schematic illustrations of jointed apparatus for bone fixation, in accordance with an embodiment of the present invention.

### DETAILED DESCRIPTION OF EMBODIMENTS OF THE INVENTION

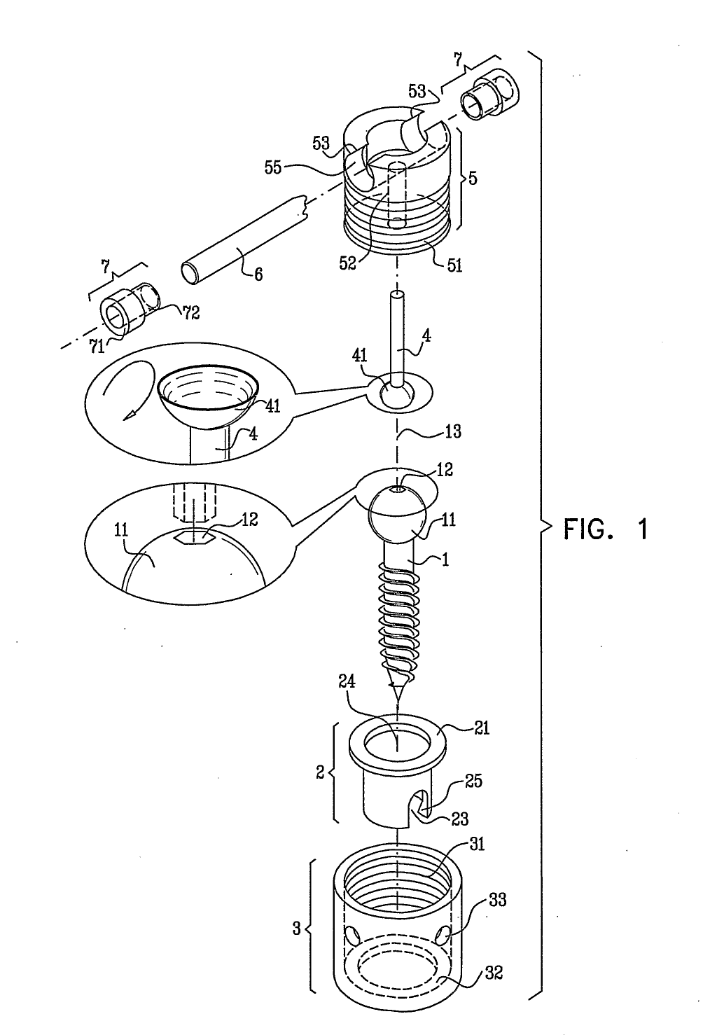

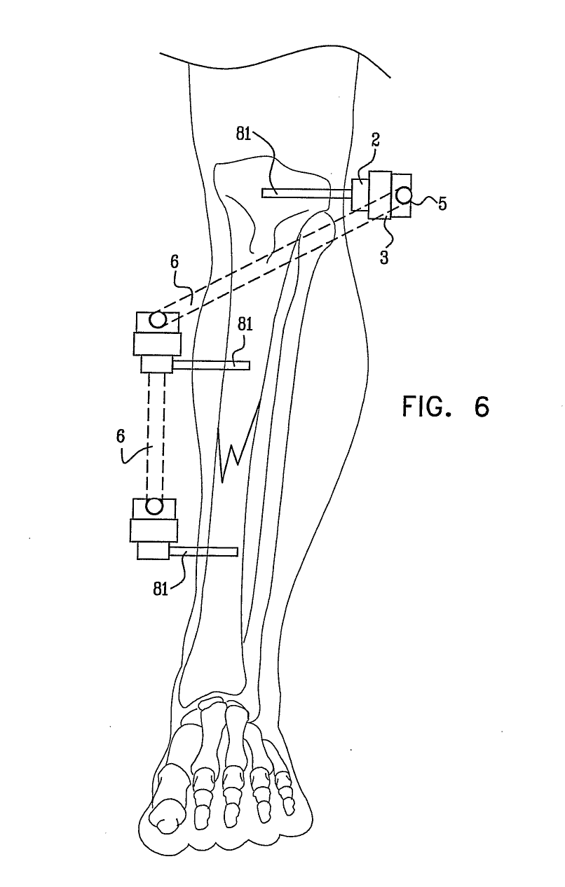

FIG. 1 is a schematic illustration of apparatus for bone fixation, in accordance with an embodiment of the present invention. The apparatus comprises an integrated double articulation element, such as a double-jointed bone fixation element, as shown in FIG. 1. A bone anchoring member, for example, a longitudinal bone insertion element such as a screw 1 or a pin (FIG. 6) is passed through a joint element such as a mobile aperture element 2 and through a retaining connector 3. During an implantation procedure, screw 1 is screwed into bone of a patient by accessing a recess 12 or other portion of the screw.

A round head 11 of screw 1 articulates with an inner surface of mobile aperture element 2. A portion of the inner surface forms a widened area 25 of mobile aperture element 2, which prevents head 11 of screw 1 from passing through the lower opening of the mobile aperture element.

Typically, an interface lip 21 of mobile aperture element 2 articulates with a support lip 32 of retaining connector 3.

Screw 1 is formed of three portions: (a) head 11, an upper segment, near head 11, which is unthreaded and is adapted to move inside an aperture 23 of mobile aperture element 2. Typically, mobile aperture element 2 is rotated around its longitudinal axis and then around another axis during the implantation procedure, such that aperture 23 thereof is aligned with respect to an axis 13 of screw 1 such that the direction of an axis 24 of mobile aperture element 2 can be changed, to cause axis 24 to diverge from axis 13 by a desired amount determined by the anatomy and/or pathology of the patient (e.g., by turning axis 24 through less than 60 degrees, 60-90 degrees, or more than 90 degrees). In addition, simultaneously, rotational mobility of mobile aperture element 2, around axis 13, is provided as well.

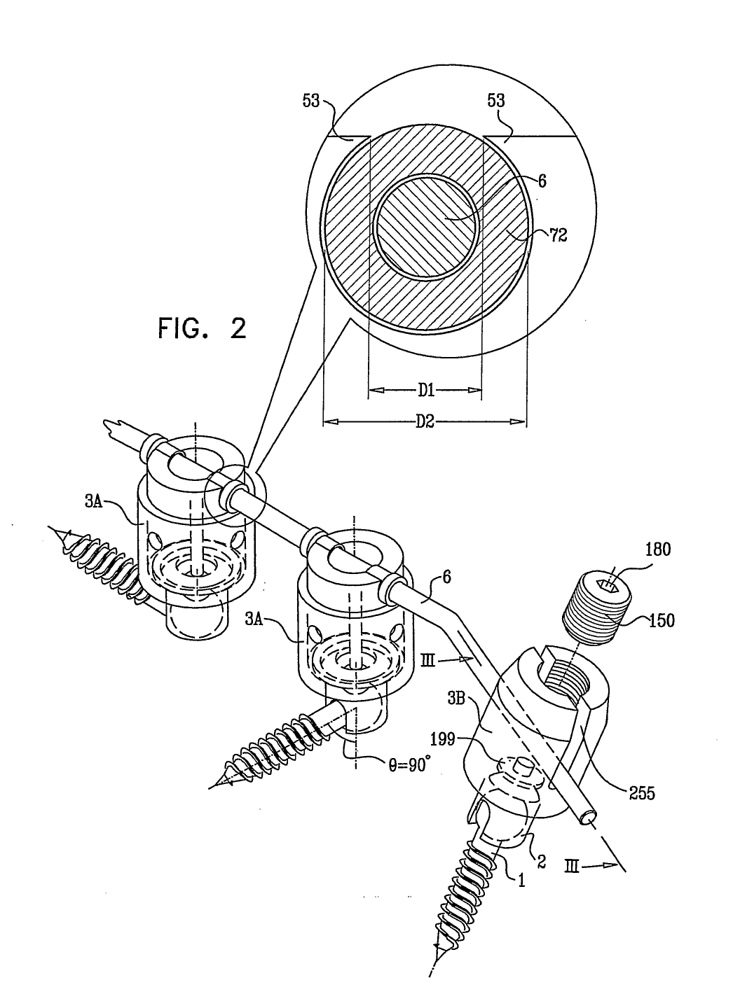

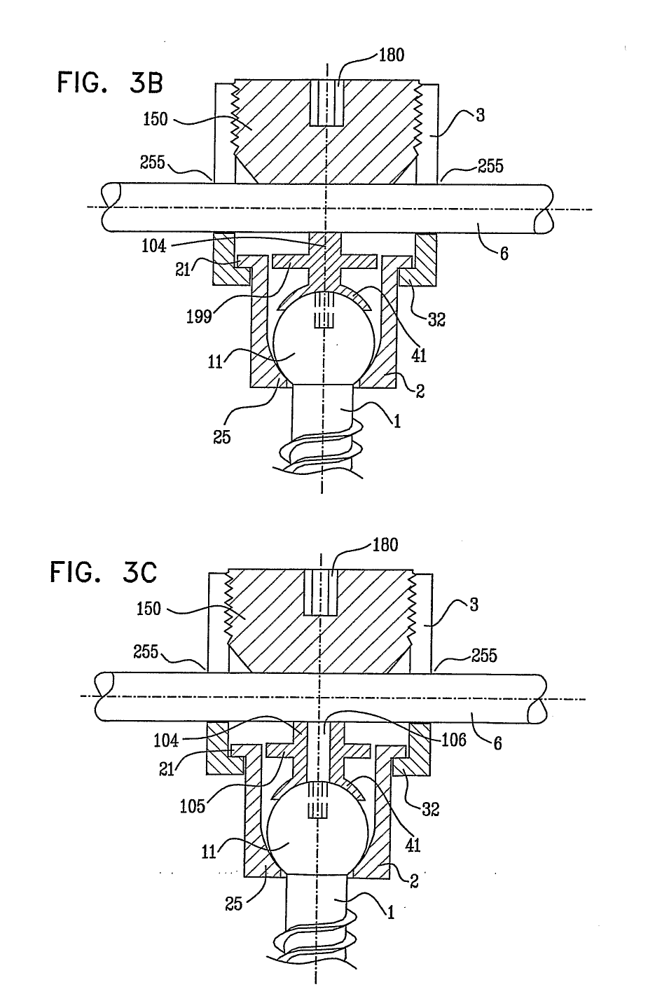

A locking pin 4, having a bowl-shaped head portion 41, is placed over round head 11 of screw 1, such that head portion 41 can rotate on round head 11. Locking pin 4 is placed within a lumen 52 of a rod holder 5. Rod holder 5 is free to rotate around its longitudinal axis prior to being rigidly coupled, via locking pin 4, to screw 1. During the implantation procedure, rod holder 5 is placed at a desired angular orientation, typically chosen such that a rod 6 lowered into rod holder 5 is oriented generally toward another bone fixation element (see FIGS. 2 and 4).

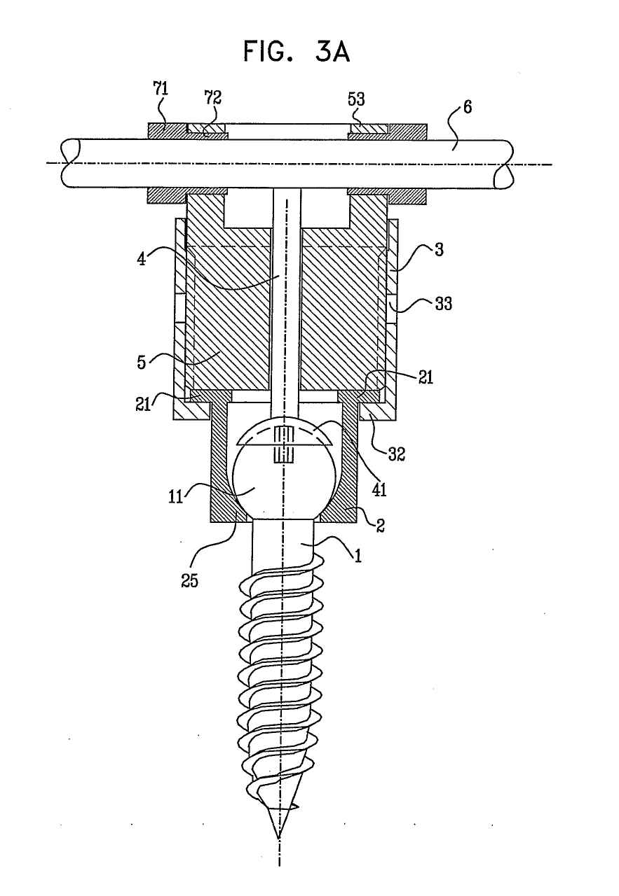

At least one rod retaining portion 7, comprising a head 71 and a body 72, is slid along rod 6. (As shown in FIG. 1, rod retaining portion 7 is distinct from rod holder 5.) Body 72 is passed into cut-outs 55 of rod holder 5, and prevented, by upper lips 53 of rod holder 5, from separating from rod holder 5 in an upward direction (i.e., upward as shown in the figure). Use of the rod retaining portion allows rod 6 and screw 1 to be rigidly coupled to each other, via intermediate components. It is to be appreciated that the scope of the present invention includes the use of other components known in the art (such as screw 150 of FIGS. 2 and 3B) for securing a rod in place, in addition to or instead of rod retaining portion 7.

When rod 6 is securely disposed within rod holder 5 (as shown in FIG. 3) and the desired angular orientation of rod holder 5 has been attained, retaining connector 3 is rotated, to push screw 1 into locking pin 4, thereby pushing locking pin 4 into rod 6. For example, one or more turning holes 33 on retaining connector 3 may be accessed, and rotated, such that a coupling mechanism, such as threads 31, of connector 3, rotates along a corresponding coupling mechanism, such as threads 51, of rod holder 5. Instead of or in addition to the use of a threaded coupling mechanism, the coupling mechanism may comprise such known mechanisms as a ratchet coupling mechanism or a click-into-place coupling mechanism.

In this manner, double-jointed control of the bone fixation apparatus is attained, namely through (1) the movement of mobile aperture element 2 with respect to screw 1, and (2) the rotational control of mobile aperture element 2 with respect to retaining connector 3. It is noted that rotation of retaining connector 3 with respect to rod holder 5 reduces the distance between the center of the double-jointed bone fixation element and the center of the rod and the rod holder. In other words, screwing retaining connector 3 on rod holder 5 brings the center of the jointed element closer to the rod and the rod holder, squeezing locking pin 4 and achieving the desired tightening and rigid fixation of the rod with respect to screw 1.

For some applications, locking pin 4 moves freely within lumen 52 of rod holder 5 prior to tightening, but is constrained to not be able to exit the lumen (or to not be able to exit the lumen easily). As appropriate, a portion of locking pin 4 and/or of one of the pieces of the apparatus has a protrusion or is otherwise shaped to provide this constraining functionality. Similarly, other locking pins usable with this or with other embodiments of the invention (e.g., locking pin 104 described hereinbelow) may also be thus constrained.

FIG. 2 is a schematic illustration of apparatus for bone fixation, as shown in FIG. 1, in which three screws 1 are positioned at different respective orientations (surrounding anatomy not shown), in accordance with an embodiment of the present invention. Rod 6 is seen to be bent, but depending on the local anatomy, could alternatively be generally straight. In any case, no significant deformations of the rod are utilized to achieve the configuration of screws 1 shown in the figure. The scope of the present invention includes using one or more non-straight rods 6 (e.g., as shown in FIG. 4), and/or one or more straight rods 6 (e.g., as shown in FIG. 6), as well as using a rod 6 that is bent during an implantation procedure to better facilitate coupling of the rod to a plurality of rod holders 5.

The exploded view in FIG. 2 shows rod 6 surrounded by body 72 of rod retaining portion 7, and thereby secured within rod holder 5. The diameter of rod 6 is less than the distance D1 between upper lips 53 of rod holder 5. Therefore, rod 6 can be lowered into its desired position within rod holder 5, prior to sliding rod retaining portion 7 under upper lips 53. Outer diameter D2 of body 72, however, is greater than distance D1, typically preventing rod 6 and body 72 from passing up and out of rod holder 5, between upper lips 53.

It is noted that FIG. 2 shows three retaining connectors 3, displayed on the page in a line from upper left to lower right. The upper left and middle retaining connectors are referred to as “retaining connectors 3A,” and are the same as retaining connector 3 shown in FIG. 1. Retaining connectors 3A are shown in cross-section in FIG. 3A. The lower right retaining connector in FIG. 2 is designated “retaining connector 3B,” and is shown (with some changes) in cross-section in FIG. 3B. A rod retaining portion (in this case, a screw 150) is screwed into the upper portion of retaining connector 3B, in order to press rod 6 down in a slot 255 of retaining connector 3B. Typically, a recess 180 of screw 150 is accessed to facilitate the screwing of screw 150.

Although FIG. 2 shows two different types of retaining connectors (3A and 3B), the scope of the present invention includes the use of only retaining connectors 3A, as well as the use of only retaining connectors 3B.

FIG. 3A is a schematic cross-sectional illustration of the bone fixation apparatus described hereinabove, after retaining connector 3 (in this case, 3A) has been screwed onto rod holder 5, in accordance with an embodiment of the present invention.

FIG. 3B is a schematic cross-sectional illustration of the bone fixation apparatus described hereinabove, after retaining connector 3 (in this case, 3B) has been coupled to rod 6 and to a locking pin 104, in accordance with an embodiment of the present invention. Screw 150 is shown fully screwed in to retaining connector 3B, such that rod 6 is rigidly held in place by being squeezed between the top surface of locking pin 104 and screw 150. In addition, bowl-shaped head portion 41 of locking pin 104 applies a downward force to round head 11 of screw 1, keeping screw 1 securely in place against mobile aperture element 2, because of the force applied to locking pin 104 by rod 6 as a result of the screwing in of screw 150.

FIG. 3C is a schematic cross-sectional illustration of the bone fixation apparatus described hereinabove, after retaining connector 3 (in this case, 3B) has been coupled to rod 6 and to locking pin 104, in accordance with an embodiment of the present invention. The apparatus shown in FIG. 3C is generally similar to that shown and described with reference to FIG. 3B, except that locking pin 104 in the embodiment shown in FIG. 3C is shaped to define a bore 106 therethrough. Bore 106 permits a surgeon to screw screw 1 into bone, by accessing recess 12 of screw 1 with a tool that is placed through bore 106. Subsequently, rod 6 is placed in slot 255, and then screw 150 is screwed downward, tightening the rod with respect to screw 1 (via intervening locking pin 104).

FIG. 4 is a schematic illustration of the bone fixation apparatus described hereinabove, in which three screws 1 are implanted in respective vertebrae of a patient, in accordance with an embodiment of the present invention. Each screw 1 is positioned at a different angle, using the double-jointed functionality of the bone fixation apparatus. For some applications, when a plurality of bone fixation elements are utilized, one or more are double jointed, and one or more are single jointed (e.g., all are double jointed, or alternating bone fixation elements are double jointed).

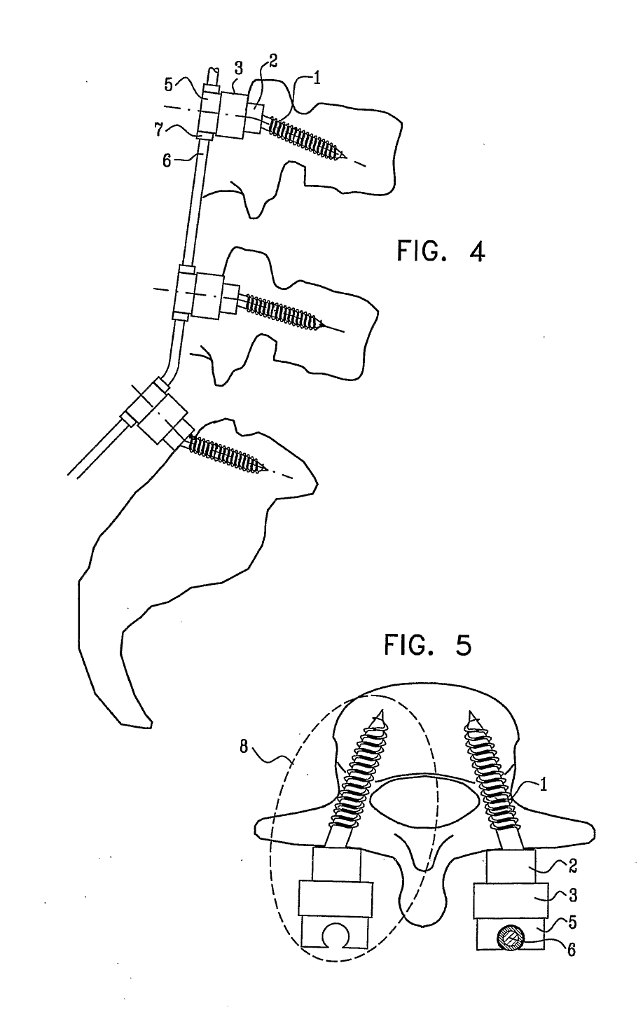

FIG. 5 is a schematic illustration of two screws 1 implanted in a vertebrae, in accordance with an embodiment of the present invention. On the left, a double-jointed bone fixation element 8 is shown, prior to its being coupled to rod 6. Double-jointed bone fixation element 8 comprises screw 1, mobile aperture element 2, retaining connector 3, locking pin 4, and rod holder 5, which typically function as described hereinabove. On the right, double-jointed bone fixation element 8 is shown after it has been coupled via body 72 to rod 6.

FIG. 6 is a schematic illustration of three pins 81 implanted in a tibia of a subject, in accordance with an embodiment of the present invention. Except for the use of pins instead of screws, the apparatus of FIG. 6 is generally similar to the apparatus described hereinabove, mutatis mutandis.

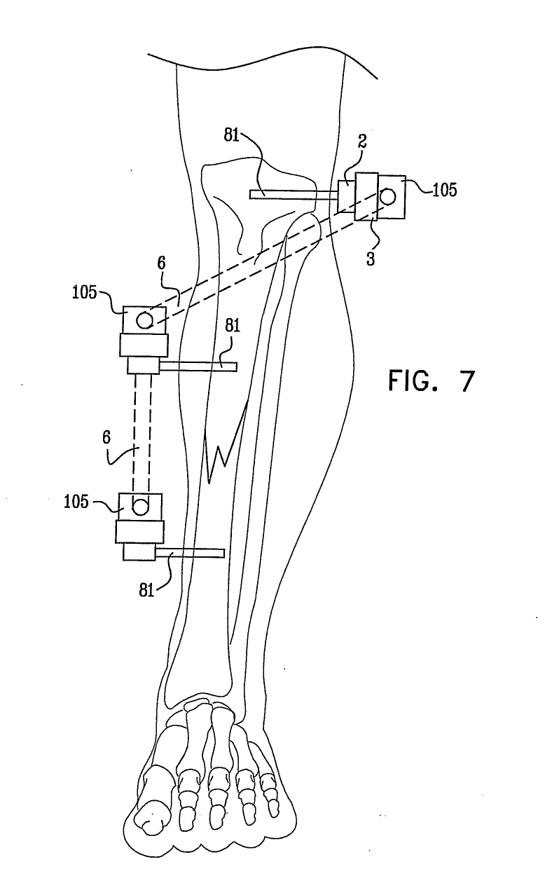

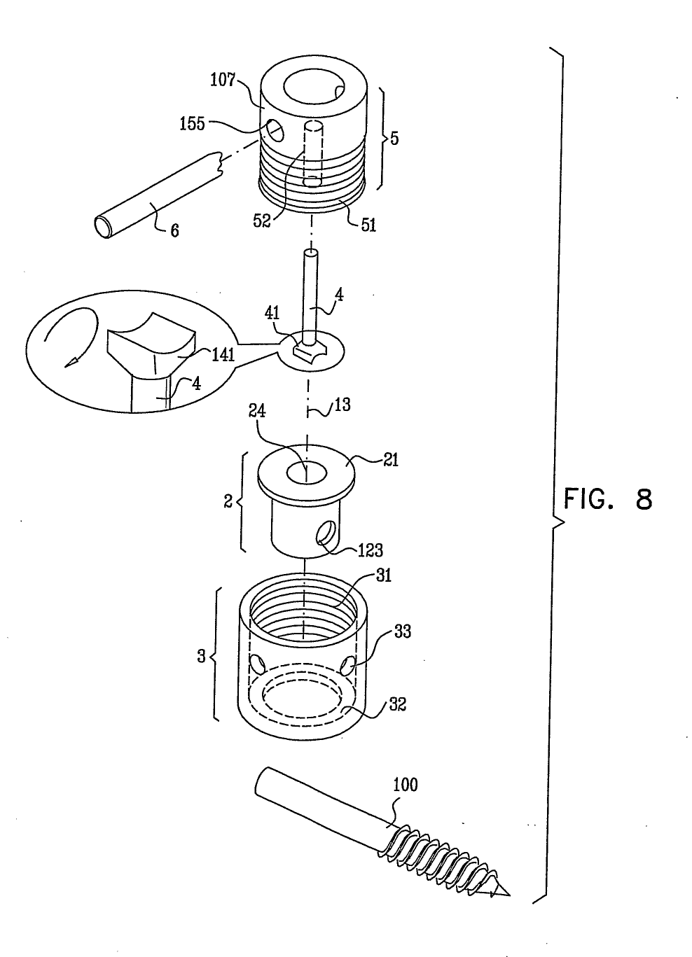

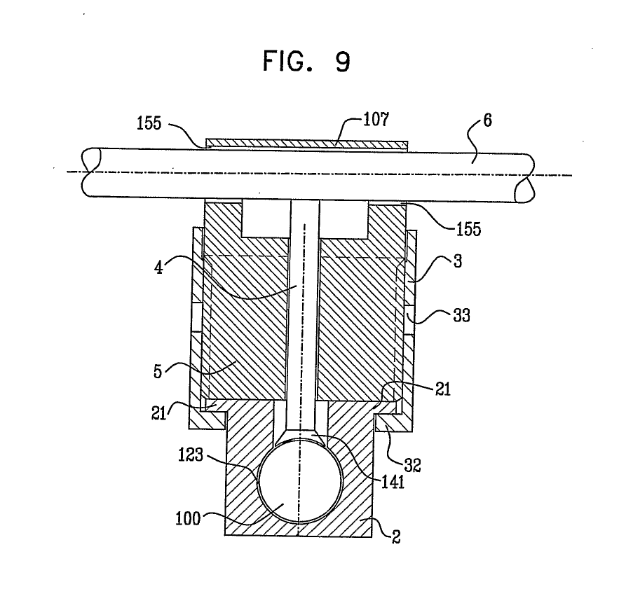

FIG. 7 is a schematic illustration of three pins 81 implanted in a tibia of a subject, in accordance with another embodiment of the present invention. In this embodiment, three jointed devices, the construction of which is shown in FIGS. 8 and 9, are used to stabilize a fracture.

FIG. 8 is a schematic illustration of jointed apparatus for bone fixation, utilizing central locking pin 4, in accordance with an embodiment of the present invention. A bone anchoring member, for example, a longitudinal bone insertion element such as a screw 100 or a pin (FIG. 6) is passed through a joint element such as mobile aperture element 2, after mobile aperture element 2 has been lowered through retaining connector 3. In the embodiment shown in FIG. 8, mobile aperture element 2 is shaped to define a hole 123 (unlike slot-like aperture 23 shown in FIG. 1). Screw 100 is passed into hole 123 when interface lip 21 of mobile aperture element 2 is in contact with support lip 32 of retaining connector 3. Articulation of lip 21 and lip 32 with respect to each other, for example, constitutes the action of a jointed element. During an implantation procedure, screw 100 is screwed into bone of a patient typically by using a chuck or other tool that grips a proximal (unthreaded) portion of screw 100. For some applications, at least part of the proximal end of screw 100 has flat sides (e.g., it is hexagonal), to facilitate the screwing of screw 100 into the bone.

In the embodiment shown in FIG. 8, locking pin 4, having a head portion 41 comprising an arch-shaped head portion 141, is placed over the unthreaded body of screw 100. Locking pin 4 is placed within a lumen 52 of rod holder 5. Rod holder 5 is free to rotate around its longitudinal axis prior to being coupled, via locking pin 4, to screw 100. During the implantation procedure, rod holder 5 is placed at a desired angular orientation, typically chosen such that rod 6 inserted into a hole 155 of rod holder 5 is oriented generally toward another bone fixation element (see FIGS. 2 and 4).

At least one rod retaining portion 107 is formed integrally to the construction of rod holder 5 (unlike rod retaining portion 7, which is shown in FIG. 1 as being distinct from rod holder 5). As shown, rod retaining portion 107 is located at the upper portion of hole 155, closing hole 155. (This is unlike the embodiment shown in FIG. 1, in which cut-outs 55 are open at their upper portions.) It is to be appreciated that the scope of the present invention includes the use of other components known in the art for securing a rod in place, in addition to or instead of rod retaining portion 107.

As appropriate based on the needs of a given procedure, screw 100 may be placed in hole 155 and rod 6 may be placed in 123.

When rod 6 is securely disposed within rod holder 5 (as shown in FIG. 9) and the desired angular orientation of rod holder 5 has been attained, retaining connector 3 is rotated, to push screw 100 into locking pin 4, thereby pushing locking pin 4 into rod 6. For example, one or more turning holes 33 on retaining connector 3 may be accessed, and rotated, such that a coupling mechanism, such as threads 31, of connector 3, rotates along a corresponding coupling mechanism, such as threads 51, of rod holder 5. Instead of or in addition to the use of a threaded coupling mechanism, the coupling mechanism may comprise such known mechanisms as a ratchet coupling mechanism or a click-into-place coupling mechanism.

It is noted that rotation of retaining connector 3 reduces the distance between the center of the jointed element and the center of the rod and the rod holder. In other words, screwing retaining connector 3 on rod holder 5 brings the center of the jointed element closer to the rod and the rod holder, squeezing locking pin 4 and achieving the desired tightening and rigid fixation of the rod with respect to screw 100.

FIG. 9 is a schematic cross-sectional illustration of the bone fixation apparatus described hereinabove with reference to FIG. 8, after retaining connector 3 has been screwed onto rod holder 5, in accordance with an embodiment of the present invention. As is seen, locking pin 4 simultaneously serves two functions: (a) locking pin 4 squeezes rod 6 against rod retaining portion 107, and (b) locking pin 4 squeezes screw 100 against mobile aperture element 2. In this manner, locking pin 4 rigidly stabilizes rod 6 with respect to screw 100 (via intermediate components).

It is to be appreciated that the various components described hereinabove may be replaced by other functionally similar components, or combinations thereof, which may be placed at different locations with respect to patient anatomy or other components of the apparatus. Similarly, the apparatus described hereinabove may be disposed in other configurations with respect to a patient's anatomy. For example, screw 1 may be placed into a non-vertebral bone, or rod 6 may be disposed outside of the patient's body, or a bone shown in a figure as being treated by particular apparatus may instead be treated by apparatus shown in a different figure.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. (canceled)

2. The apparatus according to claim 22, wherein the integrated double articulation element comprises a plurality of integrated double articulation elements, configured to be coupled to the rod.

3. The apparatus according to claim 22, wherein the apparatus further comprises a single-jointed bone fixation element, configured to be coupled to the rod.

4. The apparatus according to claim 22, wherein the rod retaining portion comprises at least one ring slidably coupled to the rod, configured to slide along the rod into a position that retains the rod in place with respect to the bone fixation element.

5. The apparatus according to claim 22, wherein the rod retaining portion comprises a screw.

6. The apparatus according to claim 31, further comprising:
a locking pin, configured to fix a position of the rod with respect to the bone anchoring member, due to rotation of the portion of the integrated double articulation element that shortens a distance between the rod and the bone anchoring member.

7-10. (canceled)

11. The apparatus according to claim 22,
wherein the integrated double articulation element comprises a mobile aperture element,
wherein the integrated double articulation element comprises a retaining connector, rotatably coupled to the mobile aperture element,
wherein at a joint of the integrated double articulation element, the retaining connector is configured to rotate around a longitudinal axis of the mobile aperture element,
wherein the rod holder is configured to be rotated to facilitate placement of the rod in a desired alignment, and
wherein the rotation of the retaining connector at the joint is configured to maintain the desired alignment of the rod holder.

12. The apparatus according to claim 11,
wherein the retaining connector, is shaped to define a support lip,
wherein the mobile aperture element is shaped to define an interface lip thereof, and
wherein the interface lip is configured to be in contact with the support lip.

13. The apparatus according to claim 12, wherein the interface lip is configured to articulate with the support lip.

14. (canceled)

15. The apparatus according to claim 22, wherein the rod holder and the rod retaining portion are formed as an integral structure.

16-17. (canceled)

18. A method, comprising:
inserting a longitudinal bone insertion element into a bone;
rotating a double-jointed element to a desired orientation thereof, the double-jointed element being coupled to the longitudinal bone insertion element;
coupling a rod holder to the double-jointed element;
identifying a desired angular orientation of the rod holder;
positioning the rod holder at the desired angular orientation;
coupling a rod to the rod holder; and
reducing motion among the rod holder, the longitudinal bone insertion element, and the rod by tightening the double-jointed element.

19. The method according to claim 18, wherein reducing the motion comprises reducing the motion via a locking pin, coupled at one end thereof to the rod holder and the rod, and at another end thereof to the longitudinal bone insertion element.

20. A method, comprising:
inserting at least a first and a second longitudinal bone insertion element into respective bone sites of a patient, at different angular orientations of insertion;
coupling the first and second longitudinal bone insertion elements to respective first and second rod holders;
reducing an angular divergence between the rod holders by (a) manipulating a first double-jointed element that is coupled to the first rod holder and to the first longitudinal bone insertion element, and (b) manipulating a second double-jointed element that is coupled to the second rod holder and to the second longitudinal bone insertion element;
coupling the first and second double-jointed elements via a rod; and
reducing motion among the first longitudinal bone insertion element, the second longitudinal bone insertion element, and the rod, by tightening the double-jointed elements.

21. The method according to claim 20, wherein reducing the motion comprises reducing the motion via a locking pin, coupled at one end thereof to the first rod holder and the rod, and at another end thereof to the first longitudinal bone insertion element.

22. Apparatus for treating bone, comprising:
a bone anchoring member;
a rod;
at rod holder configured to hold the rod;
at least one rod retaining portion for retaining the rod in position with respect to the rod holder; and
coupled to the rod holder and movable with respect to the rod holder, an integrated double articulation element that (a) provides mobility of the rod holder by being adapted to move in at least two axes relative to the bone anchoring members at a first joint, and (b) provides further mobility of the rod holder relative to the bone anchoring members at a second joint.

23. A method for treating bone, comprising:
a. providing a bone fixation system, comprising:
1. at least two bone anchoring members;
2. at least two rod holders;
3. coupled to each rod holder, an integrated double articulation element that (a) provides mobility of the rod holder by being adapted to move in at least two axes relative to one of the bone anchoring members at a first joint, and (b) provides further mobility of the rod holder relative to the one of the bone anchoring members at a second joint;
4. for each rod holder, at least one rod retaining portion; and
a connecting rod, configured to be coupled to each of the rod holders;
b. inserting the at least two bone anchoring members in at least one bone;
c. coupling each bone anchoring member to a respective one of the rod holders;
d. bringing each of the rod holders, via its respective integrated double articulation element, to an orientation suitable for coupling the rod holder to the connecting rod;
e. coupling the connecting rod to each of the rod holders via at least one rod retaining portion; and
f. tightening each of the rod holders to minimize motion between each rod holder and the respective bone anchoring member and to minimize motion between the connecting rod and each of the rod holders.

24. Apparatus, comprising:
a rod;
a rod holder shaped to define an opening through which the rod passes;
a longitudinal element configured for insertion into bone of a subject;
a jointed element which (a) facilitates motion of the rod relative to the longitudinal element, (b) is shaped to define a space therein configured for holding the longitudinal element, and (c) is configured to reduce a distance between a center of the jointed element and a center of the rod holder; and
a locking pin having first and second ends, the first end pushing the rod against the rod holder when the rod is in the opening, and the second end pushing a lateral portion of the longitudinal element against a portion of the jointed element.

25. The apparatus according to claim 24, wherein the jointed element comprises a coupling mechanism selected from the group consisting of: a threaded coupling mechanism, a ratchet coupling mechanism, and a click-into-place coupling mechanism, and wherein the selected coupling mechanism facilitates the reduction in distance between the center of the jointed element and the center of the rod holder.

26-30. (canceled)

31. The apparatus according to claim 22, wherein the integrated double articulation element and the rod holder are arranged such that a movement of a portion of the integrated double articulation element with respect to the rod holder tightens the rod with respect to the bone anchoring member.

32. The apparatus according to claim 22, wherein the bone anchoring member has a longitudinal axis, and wherein the bone anchoring member is rotatable by more than 90 degrees with respect to the integrated double articulation element, around an axis of the bone anchoring member other than the longitudinal axis of the bone anchoring member.

33. The apparatus according to claim 6, wherein rotation of the portion of the integrated double articulation element pulls the rod toward the bone anchoring member.
